(19) United States
(12) Patent Application Publication
(10) Pub. No.: US 2006/0223874 A1
(43) Pub. Date: Oct. 5, 2006

(54) Heterocyclic reverse transcriptase inhibitors

(75) Inventors: Michael Martin, San Francisco, CA (US); Harit Umesh Vora, Fort Collins, CO (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/388,541

(22) Filed: Mar. 23, 2006

Related U.S. Application Data
(60) Provisional application No. 60/664,813, filed on Mar. 24, 2005.

(51) Int. Cl. A61K 31/4196; C07D 249/12

(52) U.S. Cl. 514/384; 548/263.2

(57) ABSTRACT

The present invention provides compounds for treating or preventing an HIV infection, or treating AIDS or ARC comprising administering a compound according to formula I where R¹, R² and R³, are as defined herein.

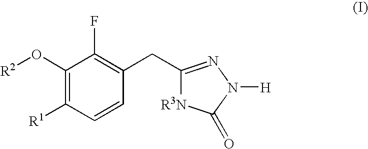

### CROSS REFERENCE TO RELATED APPLICATIONS

This application claims the benefit of priority to U.S. Ser. No. 60/664,813 filed Mar. 24, 2005 the contents of which are hereby incorporated in their entirety by reference.

### FIELD OF THE INVENTION

The invention relates to the field of antiviral therapy and, in particular, to non-nucleoside reverse transcriptase inhibitors for treating Human Immunodeficiency Virus (HIV) mediated diseases. The invention provides novel heterocyclic compounds, pharmaceutical compositions comprising these compounds, methods for treating or preventing a human immunodeficiency virus (HIV) infection, or treating AIDS or ARC employing said compounds in monotherapy or in combination therapy.

### BACKGROUND OF THE INVENTION

The human immunodeficiency virus HIV is the causative agent of acquired immunodeficiency syndrome (AIDS), a disease characterized by the destruction of the immune system, particularly of the CD4⁺ T-cell, with attendant susceptibility to opportunistic infections. HIV infection is also associated with a precursor AIDS—related complex (ARC), a syndrome characterized by symptoms such as persistent generalized lymphadenopathy, fever and weight loss.

In common with other retroviruses, the HIV genome encodes protein precursors known as gag and gag-pol which are processed by the viral protease to afford the protease, reverse transcriptase (RT), endonuclease/integrase and mature structural proteins of the virus core. Interruption of this processing prevents the production of normally infectious virus. Considerable efforts have been directed towards the control of HIV by inhibition of virally encoded enzymes.

Currently available chemotherapy targets two crucial viral enzymes: HIV protease and HIV reverse transcriptase (J. S. G. Montaner et al. Antiretroviral therapy. “the state of the art”, Biomed & Pharmacother. 1999 53:63-72; R. W. Shafer and D. A. Vuitton, Highly active retroviral therapy (HAART) for the treatment of infection with human immunodeficiency virus type 1, Biomed. & Pharmacother. 1999 53:73-86; E. De Clercq, New Developments in Anti-HIV Chemotherap. Curr. Med. Chem. 2001 8:1543-1572). Two general classes of RTI inhibitors have been identified: nucleoside reverse transcriptase inhibitors (NRTI) and non-nucleoside reverse transcriptase inhibitors (NNRTI).

NRTIs typically are 2′,3′-dideoxynucleoside (ddN) analogs which must be phosphorylated prior to interacting with viral RT. The corresponding triphosphates function as competitive inhibitors or alternative substrates for viral RT. When incorporated into nucleic acids the nucleoside analogs terminate the chain elongation process. HIV reverse transcriptase has DNA editing capabilities which enable resistant strains to overcome the blockade by cleaving the nucleoside analog and continuing the elongation. Currently clinically used NRTIs include zidovudine (AZT), didanosine (ddI), zalcitabine (ddC), stavudine (d4T), lamivudine (3TC) and tenofovir (PMPA).

NNRTIs were first discovered in 1989. NNRTI are allosteric inhibitors which bind reversibly at a nonsubstrate binding site on the HIV reverse transcriptase thereby altering the shape of the active site or blocking polymerase activity (R. W. Buckheit, Jr., Non-nucleoside reverse transcriptase inhibitors: perspectives for novel therapeutic compounds and strategies for treatment of HIV infection, Expert Opin. Investig. Drugs 2001 10(8)1423-1442; E. De Clercq The role of non-nucleoside reverse transcriptase inhibitors (NNRTIs) in the therapy of HIV-1 infection, Antiviral Res. 1998 38:153-179; G. Moyle, The Emerging Roles of Non-Nucleoside Reverse Transcriptase Inhibitors in Antiviral Therapy, Drugs 2001 61(1):19-26). Although over thirty structural classes of NNRTIs have been identified in the laboratory, only three compounds have been approved for HIV therapy: efavirenz, nevirapine and delavirdine. Although initially viewed as a promising class of compounds, in vitro and in vivo studies quickly revealed the NNRTIs presented a low barrier to the emergence of drug resistant HIV strains and class-specific toxicity. Drug resistance frequently develops with only a single point mutation in the RT.

While combination therapy with NRTIs, PIs and NNRTIs has, in many cases, dramatically lowered viral loads and slowed disease progression, significant therapeutic problems remain. The cocktails are not effective in all patients, potentially severe adverse reactions can occur and the rapidly-replicating HIV virus has proven adroit at creating mutant drug-resistant variants of wild type protease and reverse transcriptase.

4-Alkyl-5-oxo-4,5-dihydro-1H-[1,2,4]triazol-3-ylmethyl compounds have been disclosed and methods for treating or preventing a human immunodeficiency virus (HIV) infection, or treating AIDS or ARC using the same have been described by J. P. Dunn et al. in U.S. Patent Publication 20040192704 filed Mar. 23, 2004. Pyridazinone compounds have also been disclosed which are useful for the treatment of HIV infections by J. P. Dunn et al. in U.S. Patent Publication 20040198736 filed Mar. 23, 2004. Both publications are incorporated herein by reference in their entirety.

While current therapeutic options have reduced the severity of the disease and prolonged life, current therapeutic options often require complicated dosing regimens including multiple therapeutic agents and some patients experience undesirable side effects which can be sufficiently severe to restrict their use or compromise patient compliance. The emergence of resistant strains to current therapeutic options makes the development of new compounds active against resistant strains an important goal. There remains a need for safer drugs with activity against wild type and commonly occurring resistant strains of HIV.

### SUMMARY OF THE INVENTION

The present invention relates to new heterocyclic compounds which inhibit HIV reverse transcriptase, methods for treating or preventing a human immunodeficiency virus (HIV) infection, or treating AIDS or ARC by administering said compounds and pharmaceutical compositions containing said compounds admixed with at least one pharmaceutically acceptable carrier, diluent or excipient wherein said compound is a compound of formula I: 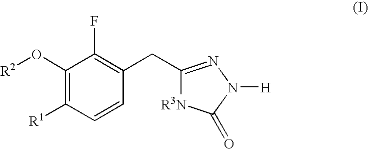
wherein R¹ is C₁₋₄ alkoxy or C₁₋₆ haloalkoxy; R² is phenyl substituted with 1 to 3 groups independently selected in each incidence from the group consisting of C₁₋₆ alkyl, C₁₋₄ haloalkyl, C₁₋₆ alkoxy, C₁₋₆ haloalkoxy, cyano, halogen; R³ is hydrogen or C₁₋₆ alkyl; and, pharmaceutically acceptable salts thereof.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition which is provided in the

### SUMMARY OF THE INVENTION

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety incorporate a hydrogen or a substituent.

In one embodiment of the present invention there is provided a compound according to formula I wherein R₁, R² and R³ are also described herein above.

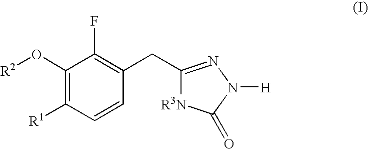

In another embodiment of the present invention there is provided a compound according to formula I wherein R¹ is C₁₋₆ alkoxy, R³ is C₁₋₄ alkyl and R² is phenyl substituted with 1 to 3 groups independently selected in each incidence from the group consisting of C₁₋₆ alkyl, C₁₋₄ haloalkyl, C₁₋₆ alkoxy, C₁₋₆ haloalkoxy, cyano, halogen.

In another embodiment of the present invention there is provided a compound according to formula I wherein R¹ is C₁₋₆ alkoxy, R³ is C₁₋₆ alkyl and R² is phenyl substituted with 1 to 3 groups independently selected in each incidence from the group consisting of C₁₋₆ haloalkyl, halogen and cyano.

In another embodiment of the present invention there is provided a compound according to formula I wherein R¹ is methoxy or ethoxy, R³ is methyl or ethyl and R² is phenyl substituted with 1 to 3 groups independently selected in each incidence from the group consisting of C₁₋₆ haloalkyl, halogen and cyano.

In another embodiment of the present invention there is provided a compound according to claim 1 wherein the compound has a structure according to formula Ia, R³ is methyl or ethyl and R⁴ is difluoromethyl, trifluoromethyl, chloro or cyano.

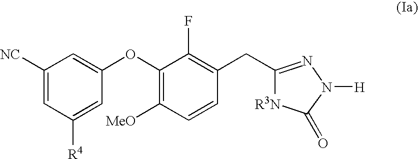

In another embodiment of the present invention there is provided a compound according to formula I wherein said formula is either 3-chloro-5-[2-fluoro-6-methoxy-3-(4-methyl-5-oxo-4,5-dihydro-1H-[1,2,4]triazol-3-ylmethyl)-phenoxy]-benzonitrile or 3-difluoromethyl-5-[2-fluoro-6-methoxy-3-(4-methyl-5-oxo-4,5-dihydro-1H-[1,2,4]triazol-3-ylmethyl)-phenoxy]-benzonitrile.

In another embodiment of the present invention there is provided a method for treating or preventing an human immunodeficiency virus (HIV) infection, or treating AIDS or ARC, in a patient in need thereof which comprises administering to the patient a therapeutically effective amount of a compound of formula I wherein R¹, R² and R³ are as described herein above.

In another embodiment of the present invention there is provided a method for treating or preventing an human immunodeficiency virus (HIV) infection, or treating AIDS or ARC, in a patient in need thereof which comprises co-administering to the patient a therapeutically effective amount of at least one compound selected from the group consisting of HIV nucleoside reverse transcriptase inhibitors, HIV nonnucleoside reverse transcriptase inhibitors, HIV protease inhibitors and viral fusion inhibitors and a therapeutically effective amount of a compound of formula I wherein R¹, R² and R³ are as described herein above.

In another embodiment of the present invention there is provided a method for treating or preventing an human immunodeficiency virus (HIV) infection, or treating AIDS or ARC, in a patient in need thereof which comprises co-administering to the patient at least one compound selected from the group consisting of efavirenz, nevirapine, delavirdine, zidovudine, didanosin, zalcitabine, stavudine, lamivudine, abacavir, adefovir, dipivoxil, saquinavir, ritonavir, nelfinavir, indinavir, amprenavir, lopinavir and T-20 in addition to a therapeutically effective amount of a compound of formula I wherein R¹, R² and R³ are as described hereinabove.

In another embodiment of the present invention there is provided a method for inhibiting a retrovirus reverse transcriptase in a patient infected with a strain of HIV or potentially exposed to a strain of HIV comprising administering to the patient a therapeutically effective amount of a compound of formula I wherein R¹, R² and R³ are as described herein above.

In another embodiment of the present invention there is provided a method for inhibiting a retrovirus reverse transcriptase in a patient infected with a strain of HIV or potentially exposed to a strain of HIV in which said retrovirus reverse transcriptase contains one mutation compared to wild type virus comprising administering to the patient a therapeutically effective amount of a compound of formula I wherein R¹, R² and R³ are as described herein above.

In another embodiment of the present invention there is provided a method for inhibiting a retrovirus reverse transcriptase in a patient infected with a strain of HIV that exhibits reduced susceptibility to efavirenz, nevirapine or delavirdine compared to wild type virus comprising administering to the patient a therapeutically effective amount of a compound of formula I wherein R¹, R² and R³ are as described herein above.

In another embodiment of the present invention there is provided a method for treating or preventing an human immunodeficiency virus (HIV) infection, or treating AIDS or ARC, in a patient in need thereof which comprises administering to the patient a therapeutically effective amount of a compound of formula I wherein R¹ is methoxy or ethoxy, R³ is methyl or ethyl and R² is as described herein above.

In another embodiment of the present invention there is provided a method for treating or preventing an human immunodeficiency virus (HIV) infection, or treating AIDS or ARC, in a patient in need thereof which comprises administering to the patient a therapeutically effective amount of a compound of formula I wherein said compound is 3-chloro-5-[2-fluoro-6-methoxy-3-(4-methyl-5-oxo-4,5-dihydro-1H-[1,2,4]triazol-3-ylmethyl]phenoxy]-benzonitrile or 3-difluoromethyl-5-[2-fluoro-6-methoxy-3-(4-methyl-5-oxo-4,5-dihydro-1H-[1,2,4]triazol-3-ylmethyl]phenoxy]-benzonitrile.

In another embodiment of the present invention there is provided a pharmaceutical composition for treating or preventing an human immunodeficiency virus (HIV) infection, or treating AIDS or ARC comprising a compound according to formula I where R¹, R² and R³ are as described herein above admixed with at least one pharmaceutically acceptable carrier, diluent or excipient.

### Definitions

“Optional” or “optionally” means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “aryloxy” as used herein denotes an O-aryl group, wherein aryl is phenyl. An aryloxy group can be unsubstituted or substituted with one or two suitable substituents. The term “phenoxy” refers to an aryloxy group wherein the aryl moiety is a phenyl ring. The term (hetero)aryloxy refers to both an aryloxy and a heteroaryloxy.

The term “cyano” as used herein refers to a carbon linked to a nitrogen by a triple bond, i.e., —C≡N.

The term “haloalkyl” as used herein denotes an unbranched or a branched chain alkyl group as defined above wherein 1, 2, 3 or more hydrogen atoms are substituted by a halogen. “C₁₋₃ haloalkyl” as used herein refers to a haloalkyl composed of 1 to 3 carbons and 1-8 halogen substituents. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “haloalkoxy” as used herein refers to a group —OR where R is haloalkyl as defined herein.

The term “halogen” or “halo” as used herein means fluorine, chlorine, bromine, or iodine. Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The present invention is contemplated to include tautomers of compounds according to formula I.

The term “wild type” as used herein refers to the HIV virus strain which possesses the dominant genotype which naturally occurs in the normal population which has not been exposed to reverse transcriptase inhibitors. The term “wild type reverse transcriptase” used herein has refers to the reverse transcriptase expressed by the wild type strain which has been sequenced and deposited in the SwissProt database with an accession number P03366.

The term “reduced susceptibility” as used herein refers to about a 10 fold, or greater, change in sensitivity of a particular viral isolate compared to the sensitivity exhibited by the wild type virus in the same experimental system

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR tradename; didanosine (ddI) available under the VIDEX tradename.; zalcitabine (ddC) available under the HIVID tradename; stavudine (d4T) available under the ZERIT trademark.; lamivudine (3TC) available under the EPIVIR tradename; abacavir (1592U89) disclosed in WO96/30025 and available under the ZIAGEN trademark; adefovir dipivoxil [bis(POM)-PMEA] available under the PREVON tradename; lobucavir (BMS-180194), a nucleoside reverse transcriptase inhibitor disclosed in EP-0358154 and EP-0736533 and under development by Bristol-Myers Squibb; BCH-10652, a reverse transcriptase inhibitor (in the form of a racemic mixture of BCH-10618 and BCH-10619) under development by Biochem Pharma; emitricitabine [(−)—FTC] licensed from Emory University under U.S. Pat. No. 5,814,639 and under development by Triangle Pharmaceuticals; β-L-FD4 (also called β-L-D4C and named β-L-2′,3′-dideoxy-5-fluoro-cytidene) licensed by Yale University to Vion Pharmaceuticals; DAPD, the purine nucleoside, (−)-β-D-2,6,-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor discovered by the NIH and under development by U.S. Bioscience Inc.

The term “non-nucleoside reverse transcriptase inhibitors” (“NNRTI”s) as used herein means non-nucleoside compounds that inhibit the activity of HIV-1 reverse transcriptase.

Typical suitable NNRTIs include nevirapine (BI-RG-587) available under the VIRAMUNE tradename; delaviradine (BHAP, U-90152) available under the RESCRIPTOR tradename; efavirenz (DMP-266) a benzoxazin-2-one disclosed in WO94/03440 and available under the SUSTIVA tradename; PNU-142721, a furopyridine-thio-pyrimide; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H,3H)-pyrimidinedione); and (+)-calanolide A (NSC-675451) and B, coumarin derivatives disclosed in U.S. Pat. No. 5,489,697

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN as well as nonpeptide protease inhibitors e.g., VIRACEPT.

Typical suitable PIs include saquinavir available in hard gel capsules under the INVIRASE tradename and as soft gel capsules under the FORTOVASE tradename; ritonavir (ABT-538) available under the NORVIR tradename; indinavir (MK-639) available under the CRIXIVAN tradename; nelfnavir (AG-1343) available under the VIRACEPT; amprenavir (141W94), tradename AGENERASE, a non-peptide protease inhibitor; lasinavir (BMS-234475; originally discovered by Novartis, Basel, Switzerland (CGP-61755); DMP-450, a cyclic urea discovered by Dupont; BMS-2322623, an azapeptide under development by Bristol-Myers Squibb, as a 2nd-generation HIV-1 PI; ABT-378; AG-1549 an orally active imidazole carbamate.

Other antiviral agents include hydroxyurea, ribavirin, IL-2, IL-12, pentafuside and Yissum Project No. 11607, Hydroxyurea (Droxia), a ribonucleoside triphosphate reductase inhibitor, the enzyme involved in the activation of T-cells. Hydroxyurea was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN (aldesleukin) tradename as a lyophilized powder for IV infusion or sc administration. IL-12 is disclosed in WO96/25171. Pentafuside (DP-178, T-20) a 36-amino acid synthetic peptide, disclosed in U.S. Pat. No. 5,464,933 and available under the FUZEON tradename; pentafuside acts by inhibiting fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Yissum Project No. 11607, a synthetic protein based on the HIV-1 Vif protein. Ribavirin, 1-β-D-ribofuranosyl-1H-1,2,4-triazole-3-carboxamide, is described in U.S. Pat. No. 4,211,771.

The term “anti-HIV-1 therapy” as used herein means any anti-HIV-1 drug found useful for treating HIV-1 infections in man alone, or as part of multidrug combination therapies, especially the HAART triple and quadruple combination therapies. Typical suitable HAART multidrug combination therapies include: (a) triple combination therapies such as two NRTIs and one PI; or (b) two NRTIs and one NNRTI; and (c) quadruple combination therapies such as two NRTIs, one PI and a second PI or one NNRTI. In treatment of naive patients, it is preferred to start anti-HIV-1 treatment with the triple combination therapy; the use of two NRTIs and one PI is preferred unless there is intolerance to PIs. Drug compliance is essential. The CD4⁺ and HIV-1-RNA plasma levels should be monitored every 3-6 months. Should viral load plateau, a fourth drug, e.g., one PI or one NNRTI could be added.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), benzyloxycarbonyl (CBZ or Z), carbonyl diimidazole (CDI), 1,4-diazabicyclo[2.2.2]octane (DABCO), diethylaminosulfur trifluoride (DAST), 1,8-diazabicyclo[5.4.0]undec-7-ene (DBU), N,N′-dicyclohexylcarbodiimide (DCC), 1,2-dichloroethane (DCE), dichloromethane (DCM), dibenzylideneacetone (dba), 1,5-diazabicyclo[4.3.0]non-5-ene (DBN), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), dimethyl sulfoxide (DMSO), (diphenylphosphino)ethane (dppe), (diphenylphosphino)ferrocene (dppf), 1-(3-dimethylaminopropyl)-3-ethylcarbodiim ide hydrochloride (EDCI), ethyl (Et), ethyl acetate (EtOAc), ethanol (EtOH), N,N-dimethylformamide (DMF), 2-ethoxy-2H-quinoline-1-carboxylic acid ethyl ester (EEDQ), diethyl ether (Et₂O), acetic acid (HOAc), 1-N-hydroxybenzotriazole (HOBt), high pressure liquid chromatography (HPLC), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), 1,1′-bis-thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA),. Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

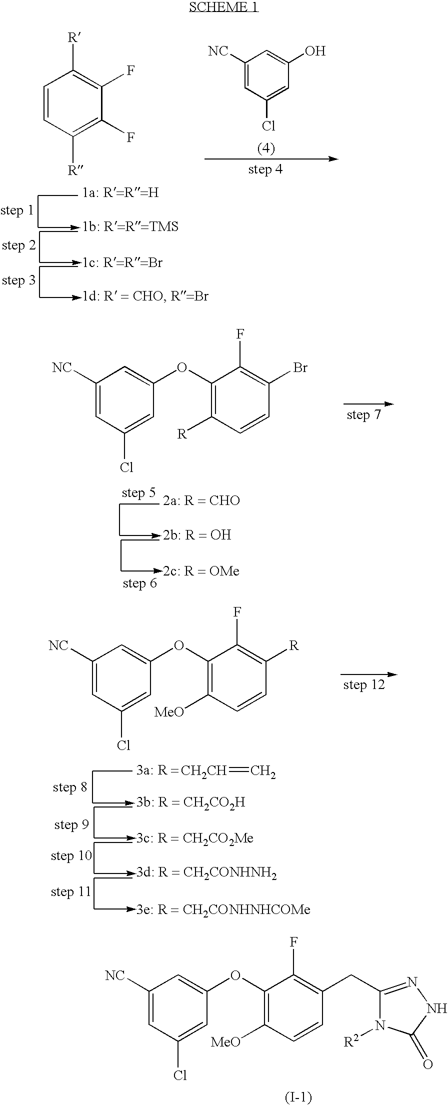

Compounds of the present invention are conveniently prepared from 4-bromo-2,3-difluoro-benzaldehyde (1d) as depicted in SCHEME 1. 4-Bromo-2,3-difluoro-benzaldehyde was conveniently prepared in 3 steps starting from ortho-difluorobenzene (1a). A mixture 1a and trimethylsilylchloride was treated with butyl lithium which afforded the disilylated product 1b. Contacting 1b with bromine afforded 1,4-dibromo-2,3-difluoro benzene (1c). Selective monometallation of 1c with iso-propylmagnesium chloride-lithium chloride complex and quenching the organomagnesium compound with DMF afforded the formyl derivative 1d.

The preparation of diaryl ethers has been reviewed (J. S. Sawyer, Recent Advances in Diaryl Ether Synthesis, Tetrahedron 2000 56:5045-5065). Introduction of the (hetero)aryloxy ether can often be accomplished by direct S_{N}Ar displacement reaction on an aromatic ring bearing a leaving group and electronegative substituents. Fluoroaromatic compounds with electronegative substituents are known to be sensitive to nucleophilic attack by soft nucleophiles. Fluorine substituents are generally significantly more labile than other halogen substituents. While hard nucleophiles like water and hydroxide fail to displace fluoride, soft nucleophiles like phenols, imidazoles, amines, thiols and some amides undergo facile displacement reactions even at room temperature (D. Boger et al., Biorg. Med. Chem. Lett. 2000 10: 1471-75; F. Terrier Nucleophilic Aromatic Displacement. The Influence of the Nitro Group VCH Publishers, New York, N.Y. 1991). Thus the reaction of 3-chloro-5-cyanophenol (4) and 1d in the presence of K₂CO₃ resulted in regiospecific displacement of the fluorine atom adjacent to the formyl substituent to afford 5a. This procedure can be use to prepare other diphenyl ethers with other substitution patterns by replacing 4 with other phenols with the desired substitution pattern.

One skilled in the art will appreciate that while SCHEME 1 illustrates the synthesis with phenol 4, other phenols within the scope of this invention are commercially available or can be prepared from available compounds by methods available in the chemical literature.

The aldehyde 5a was subjected to a Bayer-Villager oxidation with trifluoroperacetic acid (TFPAA) which underwent concomitant hydrolysis to the phenol 2b and was alkylated subsequently with Cs₂CO₃ and methyl iodide to afford the methoxy substituted analog 2c. Compounds of the present invention whereon R¹ is other than methoxy can be prepared by replacing methyl iodide with the desired alkyl iodide or alkyl triflate.

Metallation of the remaining bromine substituent with iso-PrMgCl/LiCl/THF and alkylation of the resulting arylmagnesium compound with allyl bromide afforded 3a which was oxidatively cleaved with NaIO₄/Ru(III)Cl₃ to produce the phenylacetic acid 3b. The carboxylic acid was converted to the corresponding methyl ester 3c by contacting 3b with trimethylsilyl diazomethane. The ester 3c was stirred with hydrazine to afford the corresponding hydrazide 3d which was converted to the diacylhydrazide 3e by condensation with methyl isocyanate. Compounds of the present invention wherein R³ is other then methyl can be prepared by substituting the appropriate isocyanate. The N-acyl-N-carbamoylhydrazide 3e cyclized to the triazolone I-1 upon treatment with potassium tert-butoxide in tert-butanol. Alternatively the transformation also can be accomplished with methanolic KOH.

A slightly modified reaction sequence was used to prepare I-2 (SCHEME 2). In this case, the acetic aid side chain was introduced by a Reformatsky-type reaction which directly incorporates the acetic acid in a single step. The tert-butyl ester was converted to the methyl ester and thence to the acyl hydrazide which was transformed to the triazinone by procedures analogous to those used to prepare I-1.

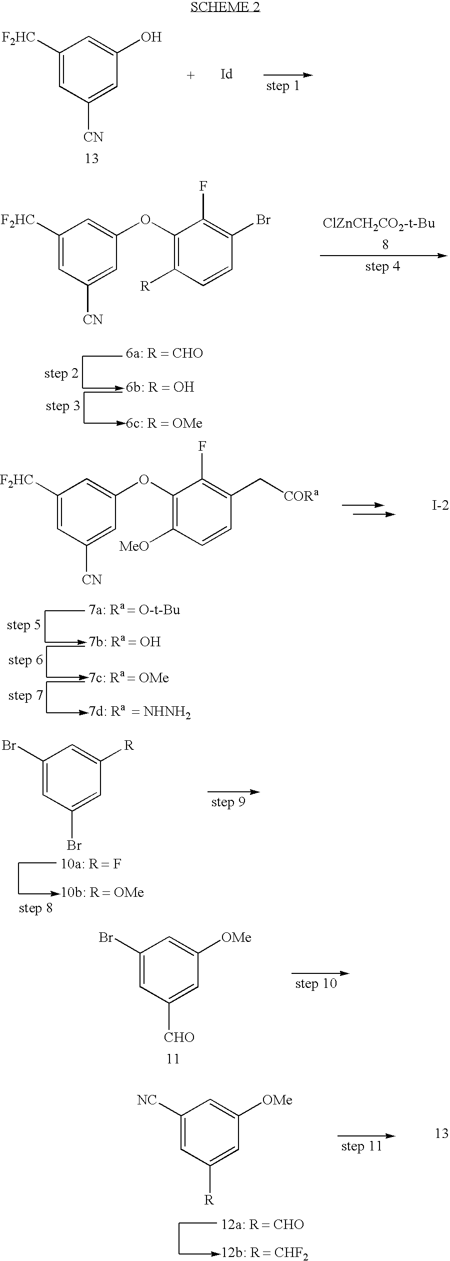

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the compound.

An example of representative compounds encompassed by the present invention and within the scope of the invention is provided in TABLE 1. These examples and preparation which follow are provided to enable those skilled in the art to more clearly understand and to practice the present invention. They should not be considered as limiting the scope of the invention, but merely as being illustrative and representative thereof.

TABLE 1

| No | Name | mp | ms |
|---|---|---|---|
| I-1 | 3-chloro-5-[2-fluoro-6-methoxy-3-(4- | 219.1-220.1 | — |
| | methyl-5-oxo-4,5-dihydro-1H-[1,2,4]triazol- | | |
| | 3-ylmethyl)-phenoxy]-benzonitrile | | |
| I-2 | 3-Difluoromethyl-5-[2-fluoro-6-methoxy-3- | 202-204 | 414 |
| | (4-methyl-5-oxo-4,5-dihydro-1H- | | |
| | [1,2,4]triazol-3-ylmethyl)-phenoxy]- | | |
| | benzonitrile | | |

### Dosage and Administration

The compounds of the present invention may be formulated in a wide variety of oral administration dosage forms and carriers. Oral administration can be in the form of tablets, coated tablets, dragees, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. Anti-HIV therapy usually includes multiple anti-HIV drugs and pharmaceutical compositions of the present invention may contain one or more other anti-HIV drugs in addition to compounds of the present invention. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The term “excipient” as used herein includes both one and more than one such excipient.

The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The preferred pharmaceutically acceptable salts are the salts formed from acetic acid, hydrochloric acid, sulfuric acid, methanesulfonic acid, maleic acid, phosphoric acid, tartaric acid, citric acid, sodium, potassium, calcium, zinc, and magnesium. It should be understood that all references to pharmaceutically acceptable salts include solvent addition forms (solvates) or crystal forms (polymorphs) as defined herein, of the same acid addition salt.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, and aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and expcipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another normucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing infections. Furthermore, treatment of a HIV infection, as used herein, also includes treatment or prophylaxis of a disease or a condition associated with or mediated by HIV infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### EXAMPLE 1

### 3-chloro-5-[2-fluoro-6-methoxy-3-(4-methyl-5-oxo-4,5-dihydro-1H-[1,2,4]triazol-3-ylmethyl]phenoxy]-benzonitrile (1-1; see SCHEME 1)

step 1—To a solution of di-iso-propylamine (150 mL, 108.3 g, 1.07 mol) in THF (500 mL) cooled to −78° C. and maintained under a N₂ atmosphere was added n-BuLi (100 mL, 1.00 mol, 10M in hexanes) over a 15 min period,. The resulting mixture was stirred for 30 min at −78° C. A mixture of 1a (45 mL, 52.110 g, 0.457 mol) and chlorotrimethylsilane (130.0 mL, 111.28 g, 1.024 mol) was added at a rate which maintained the internal reaction temperature below −50° C. The solution was stirred at −78° C. for 1 h. The reaction was quenched at −78° C. by addition of 1M H₂SO₄, diluted with MTBE and the mixture was saturated with solid NaCl. The phases were separated and the aqueous phase was extracted with MTBE (300 mL). The combined organic extracts were dried (MgSO₄), filtered and the solvents evaporated to afford 118 g (100%) of 1b as a white solid.

step 2—To neat bromine (76.9 mL, 1.50 mol) cooled to 0° C. in an ice bath was added portion wise solid 1b (126.23 g, 0.500 mol) while maintaining the internal temperature between 20-45° C. (caution: exothermic). The reaction mixture was stirred at 58° C. for 2 h. After 1 h of this period had elapsed additional bromine (45.48 g) was added and the addition funnel was rinsed with cyclohexane (10 mL). The reaction mixture was cooled to 0° C. and slowly poured into ice-cold saturated NaHSO₃ solution. After the addition the resulting mixture was saturated with solid NaCl, extracted with MTBE (500 mL and 200 mL), dried (MgSO₄) and concentrated in vacuo to afford 191 g of 1c. The reaction mixture was distilled at ca. 60 mbar which afforded 161.53 g of colorless liquid which boiled at 110° C. and contained about 11% of the monobromo derivative. The product was redistilled through a bubble ball column at ca. 50 mbar which afforded 141.3 (78.5%) of 1c with a boiling point of 93-94° C. which was >99.6 pure.

step 3—Preparation of iso-PrMgCl˜LiCl—A sample of LiCl (4.56 g, 107.6 mmol) was dried under high vacuum with a heat gun for 10 min. To the dry solid under a N₂ atmosphere at 23° C. was added iso-PrMgCl (53.8 mL, 107.6 mmol, 2M solution in THF) and the resulting mixture was stirred at 23° C. for 3 days.

To a solution of 1c (1.29 mL, 10 mmol) in THF (5 mL) at −40° C. was added the iso-PrMgCl•LiCl solution (5.5 mL, 11 mmol, 2.0M in THF) at a rate that maintained the reaction temperature below −30° C. Stirring was continued at −35 to −30° C. for 1 h then warmed to −7° C. for an additional 1 h. The reaction mixture was cooled to −30° C. and DMF (1.00 mL, 13 mmol) was added in one portion (temperature rose to −23° C.) and stirring continued for 3.5 h at −25 to +15° C. The reaction mixture was poured into 1M H₂SO₄ and ice and the resulting mixture was saturated with solid NaCl and twice extracted with MTBE. The combined extracts were dried (MgSO₄), filtered and concentrated in vacuo to afford 2.17 g (98%) of 1d as a white solid.

step 4—To a solution of 3-chloro-5-hydroxy-benzonitrile (3.84 g), K₂CO₃ powder (4.2 g) and n-butyl nitrile was added 1d (5.57 g). The reaction mixture was heated to reflux for 4.5 h when the reaction appeared complete by gc/ms. The reaction mixture was cooled and poured into water EtOAc was added. The resulting mixture was allowed to stand until the layers separated. Some crystals were present at the interface and along the walls of the upper layer which were filtered and washed with water and hexanes. The filtrate was evaporated in vacuo, the residue taken up in IPA and re-evaporated. The solid was triturated with hexane and filtered. The mother liquor was evaporated and the residue purified by SiO₂ chromatography eluting with hexane/EtOAc (80:20). The product was triturated with IPA, filtered and washed with hexanes and the product fractions combined to afford 1.45 g (83%) of 2a.

step 5—A 100 mL-round bottom flask was charged with trifluoroacetic anhydride (8.88, 4.231 mmol), cooled and stirred at 0° C. and 30% hydrogen peroxide (0.290, 8.46 mmol) was then added dropwise to the reaction vessel. The resulting solution was stirred for 2 hours at 0° C. to produce TFPAA.

To a solution of 2a (2.0, 5.64 mmol) in DCM (20 mL) stirred at 0° C. was added KH₂PO₄ (15.35 g, 112.82 mmol). To this suspension was added dropwise at 0° C. the TFPAA. The reaction was stirred for 48 h. Upon consumption of starting material reaction mixture was cooled to 0° C., and diluted with brine, and quenched with aqueous 10% sodium bisulfite. The resulting mixture was extracted with DCM and washed with brine, dried (Na₂SO₄), filtered and the solvent removed in vacuo to yield a yellow solid which was purified by SiO₂ chromatography eluting with hexane/EtOAc (92:8) to afford 1.8 g (94%) of 2b.

step 6—To a solution of 2b (1.8 g, 5.26 mmol) in DMF (15 mL) was added Cs₂CO₃ (3.43, 10.52 mmol) and iodomethane (0.74 g, 5.26 mmol). The reaction mixture was stirred at 85° C. for 12 h. When 2b was consumed, the reaction mixture was cooled to RT and the cured mixture extracted with EtOAc and the combined extracts washed with water and brine. The EtOAc was dried (Na₂SO₄), filtered and concentrated in vacuo to afford 2c as a yellow oil which was used in the next step without additional purification.

step 7—A dry 100 mL round bottom was purged with nitrogen and charged with 2c (1.6 g, 4.50 mmol) and anhydrous THF (20 mL). The mixture was cooled to −20° C. and a solution of iso-PrMgCl•LiCl (5.40 mL, 5.40 mol, 2M in THF, see step 3) was added dropwise. The reaction was stirred for 2 h at −20° C. and a solution of CuCN—LiCl (0.100 mL, 0.100 mol 1 M in THF) was added and stirred continued at −20 C. To this mixture was added allyl bromide (1.08 g, 9.0 mmol) and the mixture stirred for an additional 2 h. The reaction was quenched by addition of aqueous NH₄Cl. The mixture extracted with EtOAc and washed with water and brine. The extracts were dried (Na₂SO₄), filtered and the solvent was removed in vacuo to yield a yellow oil. The crude product was purified by SiO₂ chromatography eluting with hexane/EtOAc (95:5) to afford 1 g (70%) of 3a.

step 8—To a solution of 3a (0.100 g, 0.315 mmol), EtOAc (2 mL), MeCN (2 mL) and water (3 mL) was added NalO₄ (0.437 g, 2.050 mmol) and RuCl₃ (0.001 g, 0.006 mmol). When 3a was consumed, the crude mixture was filtered through a pad of CELITE®, washed with EtOAc and the combined EtOAc washes were washed with brine, dried (Na₂SO₄), filtered and evaporated in vacuo to afford 0.090 g (85%) of 3b as a yellow solid.

step 9—To a solution of 3b (0.216 g, 0.634 mmol) and absolute MeOH (10 mL) was added trimethylsilyldiazomethane (0.39 mL, 0.772 mmol, 2.0 M in hexanes) and stirring was continued until the acid was consumed. The reaction was quenched by the addition of HOAc and the reaction mixture partitioned H₂O and EtOAc. The aqueous phase was extracted with EtOAc and the combined EtOAc fractions were washed with water, dried (MgSO₄) and concentrated in vacuo to afford 0.14 g of 3c.

step 10—To a stirred solution of 3c (0.14 g, 0.40 mmol) and EtOH (25 mL) was added anhydrous hydrazine (0.13 mL, 4.0 mmol) and the reaction mixture was heated at reflux for 2 h. The volatile solvents were evaporated in vacuo and the crude product purified by SiO₂ column chromatography eluting with EtOAc to afford 0.117 g of 3d.

step 11—A solution of 3d (0.117 g, 0.335 mmol) and anhydrous THF (20 mL) was warmed until it became homogeneous. The reaction was allowed to cool to RT and methyl isocyanate (32.5 μL, 0.535 mmol) was added dropwise. The reaction mixture was stirred at RT for 2 h and a white precipitate formed. The reaction mixture was cooled to 0° C. and the solid filtered to afford 0.133 g of 3e as a white powder.

step 12—To a stirred mixture containing 3e (0.133 g, 0.327 mmol) and HPLC grade tert-butanol (10 mL) was added portionwise potassium tert-butoxide (4.4 mg, 0.039 mmol) and the reaction mixture was heated at reflux under an Ar atmosphere and heating was continued until starting material was consumed (ca. 3 days). Additional potassium tert-butoxide was added on two occasions after the reaction appeared to stop. The reaction mixture was cooled to RT, diluted with aqueous NH₄Cl and extracted twice with EtOAc. The combined EtOAc extracts were washed with water, dried (MgSO₄) and evaporated in vacuo. The crude product was purified by SiO₂ chromatography eluting with a DCM/MeOH gradient (2-5% MeOH) to afford 0.073 g of 1-1 as a white solid: mp 219.1-220.1° C.; Anal. Calcd. for C₁₈H₁₄ClFN₄O₃ (containing 0.1 equivalent EtOAc and 0.4 mole equivalent H₂O: C, 54.59; H, 3.88; N, 13.84, Found: C, 54.53; H, 3.60; N, 14.00.

### EXAMPLE 2

### 3-Difluoromethyl-5-[2-fluoro-6-methoxy-3-(4-methyl-5-oxo-4,5-dihydro-1H-[1,2,4]triazol-3-ylmethyl)-phenoxy]-benzonitrile (1-2; see SCHEME 2)

step 1—A suspension of 13 (1.250 g, 7.39 mmol), K₂CO₃ (1.073 g, 7.76 mmol) and butyronitrile (3 mL) was stirred and heated at 60° C. for 1 h. A solution of 1d (1.470 g, 6.65 mmol) in butyronitrile (2 mL) was added and the resulting mixture was stirred at 80° C. for 3 h. HPLC analysis indicated only partial reaction. The solution was heated to 90° C. for 1 h, then at 80° C. for 2 additional h and finally at RT overnight. The solvent was evaporated and the residue partitioned between H₂O/Et₂O/EtOAc.

The organic phase was dried and evaporated and the resulting yellow solid was triturated with 25% EtOAc/Et₂O which afforded 1.35 g of 6a. The filtrate was chromatographed on SiO₂ which afforded an additional 0.300 g of 6a (60.3% total yield).

step 2—Preparation of trifluoroperacetic acid (TFPAA)—A small vial was flushed with N₂ and trifluoroacetic anhydride (0.70 mL, 0.005 mol) was added and the liquid was cooled to 0° C. 30% aqueous H₂O₂ (0.11 mL, 0.001 mol) was added dropwise, the solution was capped and aged at 0° C. for 2 h.

The TFPAA solution was added to an ice-cold suspension of 6a (0.250 g, 0.001 mol), KH₂PO₄ (1.838 g, 0.014 mol) in DCM (3.5 mL). The vial was rinsed with a small quantity of DCM and the resulting reaction mixture was stirred at 0° C. for 2 h. The reaction was quenched with 10% sodium bisulfite and the solvent evaporated to afford ca. 250 mg of an oil which appeared to be a formate ester. The crude formate ester was dissolved in THF (4 mL) and H₂O (1 mL) and the resulting solution cooled to 0° C. Lithium hydroxide monohydrate (0.085 g, 0.002 mol) was added and the solution stirred at 0° C. for 30 min. The solution was acidified with 5% HCl and extracted with Et₂O. The solution was dried (Na₂SO₄), filtered and evaporated to afford ca. 200 mg of an oil containing a small impurity by TLC. The oil was purified on a SiO₂ flash column eluting with a EtOAc/hexane gradient (0 to 25% EtOAc) which afforded ca. 175 mg (72.3%) of 6b as a white solid insoluble in CHCl₃.

step 3—A 10 mL RB flask was flushed with N₂ and charged with the phenol 6b from step 2 (0.120 g). Dry DMF (2.0 mL) was added followed by one portion of Cs₂CO₃ (0.164 g, 0.001 mol) then MeI (0.02 mL) was added dropwise. The resulting solution was slowly heated to 80° C. and stirred for 2 h. The reaction mixture was cooled to RT and quenched carefully with 5% HCl. The resulting solution was diluted with (1:1) EtOAc/hexane, washed with water and brine, dried (MgSO₄), filtered and evaporated to afford 0.097 g (97.03%) of 6c.

step 4—An oven-dried 250 mL was cooled under N₂ and charged with 6c (1.830 g, 0.005 mol), P(O-tert-Bu)₃ and dioxane (10.0 mL). A Et₂O solution of 8 (17.70 mL, 0.5M in Et₂O; Rieke Metals, Inc.) was added dropwise and the solution was stirred at RT for 1.5 h then for 3 h at 40° C. The reaction mixture was poured into cold aqueous NH₄Cl. The resulting solution was extracted with Et₂O, washed with brine, dried (MgSO₄) and evaporated. The crude product was purified by SiO₂ chromatography eluting with an EtOAc/hexane gradient (0 to 35% EtOAc) which afforded 1.6 g (82.3%) of 7a.

step 5—The tert-butyl ester 7a (1.650 g, 4 mmol) was dissolved in DCM (20 mL) and cooled to 0° C. TFA (20 mL) was added dropwise and after the addition was completed the reaction was allowed to warm slowly to RT. The volatile solvents were evaporated, 30 mL of toluene was added and re-evaporated which afforded 1.53 g of 7b as a yellow solid which was used directly in the next step.

step 6—To a solution of 7b (1.550 g, 4 mmol) and MeOH (40 mL) cooled to 0° C. was added dropwise TMS-diazomethane ((2.0 M in DCM). When the yellow color persisted, the reaction was stirred for 10 min then quenched with several drops of HOAc. The volatile solvents were evaporated and the residue purified by SiO₂ chromatography eluting with a EtOAc/hexane gradient 5 to 50% EtOAc) to afford 1.0 g (62%) of 7c.

step 7—To a solution of 7c (0.60 g, 0.002 mol) in anhydrous EtOH (9.0 mL) maintained under an N₂ atmosphere was added anhydrous hydrazine (0.54 mL, 0.547 g, 0.017 mol) and the reaction was heated to 80-90° C. for 3 h. The solvents were evaporated and crystalline solid was washed with Et₂O to afford 0.510 g (81.7%) of 7d.

Using the procedure described in steps 11 and 12 of example 1, the hydrazide 7d was converted to the triazinone 1-2: Anal: Cal'd for C₁₉H₁₅N₄O₃F₃: C, 56.41, H, 3.45 N, 13.72; Found: C, 56.41, H, 3.45, N, 13.89.

### Preparation of 3-cyano-5-difluoromethyl-phenol

step 8—A solution of 10a, sodium methoxide (1 equivalent) and DMF were stirred overnight under an N₂ atmosphere at RT. The volatile solvents were removed in vacuo and the residue partitioned between Et₂O and water. The organic phase was washed with 5% NaOH, water and brine, dried (MgSO₄), filtered and evaporated to afford 10b.

step 9—To a solution of 10b (60 g, 0.2256 mol) and anhydrous Et₂O (1 L) cooled to −78° C. and maintained under an Ar atmosphere was added dropwise over 30 min n-BuLi (100 mL, 0.2482 mol, 2.5M in hexane). The yellow solution was stirred at −78° C. for 20 min. To the reaction mixture was added dropwise dry DMF (19 mL, 248.2 mmol) over 15 min and the reaction stirred at −78° C. for 10 min before the cooling bath was removed and the reaction allowed to warm to −30° C. over 30 min. The reaction vessel was placed in an ice-water bath and warmed to −10° C. The mixture was slowly added to an ice cold saturated aqueous NH₄Cl solution (400 mL). The organic layer was separated and the aqueous phase thrice extracted with Et₂O. The combined extracts were washed with water, dried (MgSO₄), filtered and evaporated to afford an oil which solidified on standing. The crude product was purified by SiO₂ chromatography eluting with a hexane/EtOAc gradient (3 to 5% EtOAc) to afford 11.

step 10—Cyanation of 11 to afford 12a was carried out with Zn(CN)₂, Pd(PPh₃)₄(O) and DMF. A solution of 11 (1 mmol) in DMF (2 mL) is added to a round bottomed flask containing Zn(CN)₂ (0.7 equivalents), Pd(PPh₃)₄(O) (0.2 equivalents) in DMF (15 mL). The reaction is stirred at 90° C. under an atmosphere of argon for 48 h. The reaction mixture is cooled and evaporated to dryness. The crude residue is dissolved in EtOAc, washed with brine solution, dried (MgSO₄) and evaporated. The crude product is purified by SiO₂ chromatography

step 11—DAST (21.04 mL, 519 mmol) was added to a solution of 12a (15.1 g, 94 mmol) in DCM (100 mL) under nitrogen contained in a NALGENE® bottle. EtOH (0.013 mL, 0.23 mmol) was added, and the mixture was stirred for 16 h. The reaction mixture was then added slowly to aqueous saturated NaHCO₃. After the bubbling ceased, DCM (50 mL) was added and the layers were separated. The organic layer was washed with brine (30 mL) and dried (MgSO₄). The solvent was removed and the crude product was purified by two flash chromatographies on SiO₂ eluting with an EtOAc/hexanes gradient (0% to 10% EtOAc) to afford 12b as a white solid.

### EXAMPLE 3

### HIV Reverse Transcriptase Assay: Inhibitor IC₅₀ Determination

HIV-1 RT assay was carried out in 96-well Millipore MultiScreen MADVNOB50 plates using purified recombinant enzyme and a poly(rA)/oligo(dT)₁₆ template-primer in a total volume of 50 μL. The assay constituents were 50 mM Tris/HCl, 50 mM NaCl, 1 mM EDTA, 6 mM MgCl₂, 5 μM dTTP, 0.15 μCi [³H] dTTP, 5 μg/ml poly (rA) pre annealed to 2.5 μg/ml oligo (dT)₁₆ and a range of inhibitor concentrations in a final concentration of 10% DMSO. Reactions were initiated by adding 4 nM HIV-1 RT and after incubation at 37° C. for 30 min, they were stopped by the addition of 50 μl ice cold 20% TCA and allowed to precipitate at 4° C. for 30 min. The precipitates were collected by applying vacuum to the plate and sequentially washing with 3×200 μl of 10% TCA and 2×200 μl 70% ethanol. Finally, the plates were dried and radioactivity counted in a Packard TopCounter after the addition of 25 μl scintillation fluid per well. IC_{50's} were calculated by plotting % inhibition versus log₁₀ inhibitor concentrations. (TABLE 2)

TABLE 2

| | IC50 (μM) |
|---|---|
| Compound | Wild Type HIV-RT |
| I-1 | 0.0456 |
| I-2 | 0.0129 |

### EXAMPLE 4

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium | 0.5% |
| stearate | |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

The features disclosed in the foregoing description, or the following claims expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I: 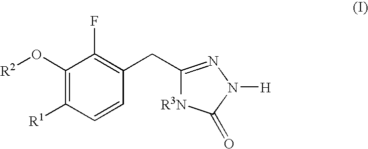
wherein
R¹ is C₁₋₆ alkoxy or C₁₋₆ haloalkoxy;
R² is phenyl substituted with 1 to 3 groups independently selected in each incidence from the group consisting of C₁₋₄ alkyl, C₁₋₆ haloalkyl, C₁₋₄ alkoxy, C₁₋₆ haloalkoxy, cyano and halogen;
R³ is hydrogen or C₁₋₆ alkyl; and,
pharmaceutically acceptable salts thereof.

2. A compound according to claim 1 wherein R¹ is C₁₋₄ alkoxy and R³ is C₁₋₆ alkyl.

3. A compound according to claim 2 wherein R² is phenyl independently substituted with C₁₋₆ haloalkyl, halogen and cyano.

4. A compound according to claim 3 wherein R¹ is methoxy or ethoxy and R³ is methyl or ethyl.

5. A compound according to claim 4 said compound having a structure according to formula Ia 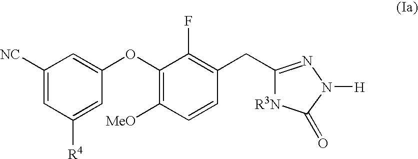
wherein R⁴ is difluoromethyl, trifluoromethyl, chloro or cyano.

6. A compound according to claim 5 wherein the compound is 3-chloro-5-[2-fluoro-6-methoxy-3-(4-methyl-5-oxo-4,5-dihydro-1H-[1,2,4]triazol-3-ylmethyl)-phenoxy]-benzonitrile.

7. A compound according to claim 5 wherein the compound is 3-difluoromethyl-5-[2-fluoro-6-methoxy-3-(4-methyl-5-oxo-4,5-dihydro-1H-[1,2,4]triazol-3-ylmethyl)-phenoxy]-benzonitrile.

8. A method for treating or preventing an human immunodeficiency virus (HIV) infection, or treating AIDS or ARC, in a patient in need thereof which comprises administering to the patient a therapeutically effective amount of a compound according to claim 1.

9. A method according to claim 8 further comprising co-administering at least one compound selected from the group consisting of HIV nucleoside reverse transcriptase inhibitors, HIV nonnucleoside reverse transcriptase inhibitors, HIV protease inhibitors and viral fusion inhibitors.

10. A method according to claim 9 wherein the non-reverse transcriptase inhibitor is selected from the group consisting of efavirenz, nevirapine and delavirdine; and/or the nucleoside reverse transcriptase inhibitor is selected from the group consisting of zidovudine, didanosin, zalcitabine, stavudine, lamivudine, abacavir, adefovir and dipivoxil; and/or the protease inhibitor is selected from the group consisting of saquinavir, ritonavir, nelfinavir, indinavir, amprenavir and lopinavir; and/or the vial fusion inhibitor is T20 (FUZEON®).

11. A method for inhibiting a retrovirus reverse transcriptase comprising administering a compound according to claim 1.

12. A method according to claim 11 wherein said retrovirus reverse transcriptase exhibits at least one mutation compared to wild type virus.

13. A method according to claim 8 wherein said patient is infected with at least one strain of HIV that exhibits reduced susceptibility to efavirenz, nevirapine or delavirdine.

14. A method according to claim 8 wherein said compound is a compound as in claim 4.

15. A method according to claim 8 wherein said compound is 3-chloro-5-[2-fluoro-6-methoxy-3-(4-methyl-5-oxo-4,5-dihydro-1H-[1,2,4]triazol-3-ylmethyl)-phenoxy]-benzonitrile or 3-difluoromethyl-5-[2-fluoro-6-methoxy-3-(4-methyl-5-oxo-4,5-dihydro-1H-[1,2,4]triazol-3-ylmethyl)-phenoxy]-benzonitrile.

16. A pharmaceutical composition for treating or preventing an human immunodeficiency virus (HIV) infection, or treating AIDS or ARC comprising a compound according to claim 1 admixed with at least one pharmaceutically acceptable carrier, diluent or excipient.
